SEQUENCE LISTING (7 sequences)

SEQ ID NO: 1 - protein, 18 aa, Homo sapiens
GLYSERGLYGLYVALASP

SEQ ID NO: 2 - protein, 1127 aa, Homo sapiens
MDEEELIQLIIEKTGKSREEIEKMVEEKIKAFNNLISRRGALLLVAKKLGVLYKNTPKEKKIGELESWEYVKVKGKILKSFGLISYSKGKFQPIILGDETGTIKAIIWNTDKELPENTVIEAIGKTKINKKTGNLELHIDSYKILESDLEIKPQKQEFVGICIVKYPKKQTQKGTIVSKAILTSLDRELPVVYFNDFDWEIGHIYKVYGKLKKNIKTGKIEFFADKVEEATLKDLKAFKGEADGSGGVDLKNKLVLIDGNSVAYRAFFALPLLHNDKGIHTNAVYGFTMMLNKILAEEQPTHILVAFDAGKTTFRHETFQDAKGGRQQTPPELSEQFPLVRELLKAYRIPAYELDHYEADDIIGTMAARAEREGFAVKVISGDRDLTQLASPQVTVEITKKGITDIESYTPETVVEKYGLTPEQIVDLKGLMGDKSDNIPGVPGIGKKTAVKLLKQFGTVENVLASIDEIKGEKLKENLRQYRDLALLSKQLAAICRDAPVELTLDDIVYKGEDREKVVALFQELGFQSFLDKMAVQTDEGEKPLAGMDFAIADSVTDEMLADKAALVVEVVGDNYHHAPIVGIALANERGRFFLRPETAVADPKFLAWLGDETKKKTMFDSKRAAVALNGKGIELAGVGVVFDLLLAAYLLDPAQAAGDVAAVAKMHQYEAVRSDEAVYGKGAKRTVPDEPTLAEQLVRKAAAIWALEEPLMDELRRNEQDRLLTELEHALAGILANMEFTGVKVDTKRLEQMGAELTEQLQAVERRIYELAGQEFNINSPKQLGTVLFDKLQLPVLKKTKTGYSTSADVLEKLAPHHEIVEHILHYRQLGKLQSTYIEGLLKVVHPVTGKVHTMFNQALTQTGRLSSVEPNLQNIPIRLEEGRKIRQAFVPSEPDWLIFAADYSQIELRVLAHIAEDDNLIEAFRRWLDIHTKTAMDIFHVSEEDVTANMRRQAKAVNFGIVYGISDYGLAQNLNITRKEAAEFIERYFASFPGVKQYMDNIVQEAKQKGYVTTLLHRRRYLPDITSRNFNVRTFAERTAMNTPIQGSAADIIKKAMIDLSVSVREERLQARLLLQGHDELILEAPKEEIGRLCRLVPEVMEQAVTLRVPLKVDYHYGPTWYDAK

SEQ ID NO: 3 - protein, 816 aa, Homo sapiens
MDEEELIQLIIEKTGKSREEIEKMVEEKIKAFNNLISRRGALLLVAKKLGVLYKNTPKEKKIGELESWEYVKVKGKILKSFGLISYSKGKFQPIILGDETGTIKAIIWNTDKELPENTVIEAIGKTKINKKTGNLELHIDSYKILESDLEIKPQKQEFVGICIVKYPKKQTQKGTIVSKAILTSLDRELPVVYFNDFDWEIGHIYKVYGKLKKNIKTGKIEFFADKVEEATLKDLKAFKGEADGSGGVDLADKAALVVEVVGDNYHHAPIVGIALANERGRFFLRPETAVADPKFLAWLGDETKKKTMFDSKRAAVALNGKGIELAGVGVVFDLLLAAYLLDPAQAAGDVAAVAKMHQYEAVRSDEAVYGKGAKRTVPDEPTLAEQLVRKAAAIWALEEPLMDELRRNEQDRLLTELEHALAGILANMEFTGVKVDTKRLEQMGAELTEQLQAVERRIYELAGQEFNINSPKQLGTVLFDKLQLPVLKKTKTGYSTSADVLEKLAPHHEIVEHILHYRQLGKLQSTYIEGLLKVVHPVTGKVHTMFNQALTQTGRLSSVEPNLQNIPIRLEEGRKIRQAFVPSEPDWLIFAADYSQIELRVLAHIAEDDNLIEAFRRWLDIHTKTAMDIFHVSEEDVTANMRRQAKAVNFGIVYGISDYGLAQNLNITRKEAAEFIERYFASFPGVKQYMDNIVQEAKQKGYVTTLLHRRRYLPDITSRNFNVRTFAERTAMNTPIQGSAADIIKKAMIDLSVSVREERLQARLLLQGHDELILEAPKEEIGRLCRLVPEVMEQAVTLRVPLKVDYHYGPTWYDAK

SEQ ID NO: 4 - protein, 663 aa, Homo sapiens
MDEEELIQLIIEKTGKSREEIEKMVEEKIKAFNNLISRRGALLLVAKKLGVLYKNTPKEKKIGELESWEYVKVKGKILKSFGLISYSKGKFQPIILGDETGTIKAIIWNTDKELPENTVIEAIGKTKINKKTGNLELHIDSYKILESDLEIKPQKQEFVGICIVKYPKKQTQKGTIVSKAILTSLDRELPVVYFNDFDWEIGHIYKVYGKLKKNIKTGKIEFFADKVEEATLKDLKAFKGEADGSGGVDLELRRNEQDRLLTELEHALAGILANMEFTGVKVDTKRLEQMGAELTEQLQAVERRIYELAGQEFNINSPKQLGTVLFDKLQLPVLKKTKTGYSTSADVLEKLAPHHEIVEHILHYRQLGKLQSTYIEGLLKVVHPVTGKVHTMFNQALTQTGRLSSVEPNLQNIPIRLEEGRKIRQAFVPSEPDWLIFAADYSQIELRVLAHIAEDDNLIEAFRRWLDIHTKTAMDIFHVSEEDVTANMRRQAKAVNFGIVYGISDYGLAQNLNITRKEAAEFIERYFASFPGVKQYMDNIVQEAKQKGYVTTLLHRRRYLPDITSRNFNVRTFAERTAMNTPIQGSAADIIKKAMIDLSVSVREERLQARLLLQGHDELILEAPKEEIGRLCRLVPEVMEQAVTLRVPLKVDYHYGPTWYDAK

SEQ ID NO: 5 - DNA, 3384 bp, Homo sapiens
atggatgaagaggaactaatacaactaataatagaaaaaactggcaaatctcgagaggaaatagaaaaaatggtggaagaaaaaattaaagcttttaacaatttaatatctcgtaggggggctttactattagtagcaaaaaaacttggtgttttgtataaaaacactccgaaagagaaaaaaattggcgaattagaaagctgggaatatgtaaaagtaaagggcaaaattctcaaatcttttggattaattagttattcgaaagggaaattccaacctattattttaggagacgaaaccggtactattaaagctattatttggaataccgataaagaattacctgaaaacactgtaatagaagctattgggaaaaccaaaattaataagaaaactggcaatttagaattacatatagacagttataaaattttagaaagcgatttagagataaaaccccaaaagcaagaatttgttgggatttgcatagttaaatatccaaaaaaacaaacccaaaaaggcacaatagtatcgaaagcaattttaactagcttagatagggaattgcctgtagtatatttcaacgattttgattgggaaataggccatatatataaagtatatggaaagcttaagaaaaacataaaaactggtaaaatagaatttttcgctgacaaagttgaggaagcaacattaaaagatctaaaagcttttaaaggagaggccgatggaagcggaggggtcgacttgaaaaacaagctcgtcttaattgacggcaacagcgtggcgtaccgcgccttttttgcgttgccgcttttgcataacgataaagggattcatacgaacgcagtctacgggtttacgatgatgttaaacaaaattttggcggaagagcagccgacccacattctcgttgcgtttgacgccgggaaaacgacgttccgccatgaaacgttccaagacgccaaaggcgggcggcagcagacgccgccggaactgtcggaacagtttccgctcgtgcgcgaattgctcaaagcgtaccgcatccccgcctatgagctcgaccattatgaagcggatgacatcatcggaacgatggcggcgcgggctgagcgagaagggtttgcagtgaaagtcatttccggcgaccgcgatttaacccagcttgcttccccgcaagtgacggtggagattacgaaaaaagggattaccgacatcgagtcgtacacgccggagacggtcgtggaaaaatacggcctcaccccggagcaaattgtcgacttgaaaggattgatgggcgacaaatccgacaacatccctggcgtgcccggcatcgggaaaaaaacagccgtcaagctgctcaagcaattcggcacggtcgaaaacgtactggcatcgatcgatgagatcaaaggggagaagctgaaagaaaatttgcgccaataccgggatttggcgcttttaagcaaacagctggccgctatttgccgcgacgccccggttgagctgacgctcgatgacattgtctacaaaggagaagaccgggaaaaagtggtcgccttgtttcaggagctcggattccagtcgtttctcgacaagatggccgtccaaacggatgaaggcgaaaagccgctcgccgggatggattttgcgatcgccgacagcgtcacggacgaaatgctcgccgacaaagcggccctcgtcgtggaggtggtgggcgacaactatcaccatgccccgattgtcgggatcgccttggccaacgaacgcgggcggtttttcctgcgcccggagacggccgtcgccgatccgaaatttctcgcttggcttggcgatgagacgaagaaaaaaacgatgtttgattcaaagcgggcggccgtcgcgctaaatgggaaaggaatcgaactggctggcgtcggcgtcgtgttcgatctgttgctggccgcttacttgctcgatccggcgcaggcggcgggcgacgttgccgcggtggcgaaaatgcatcagtacgaggcggtgcgatcggatgaggcggtctatggaaaaggagcgaagcggacggttcctgatgaaccgacgcttgccgagcagctcgtccgcaaggcggcggccatttgggcgcttgaagagccgttgatggacgaactgcgccgcaacgaacaagatcggctgctgaccgagctcgaacacgcgctggctggcattttggccaatatggaatttactggagtgaaagtggacacgaagcggcttgaacagatgggggcggagctcaccgagcagctgcaggcggtcgagcggcgcatttacgaactcgccggccaagagttcaacattaactcgccgaaacagctcgggacggttttatttgacaagctgcagctcccggtgttgaaaaagacaaaaaccggctattcgacttcagccgatgtgctagaaaagcttgcaccgcaccatgaaatcgtcgaacatattttgcattaccgccaactcggcaagctgcagtcaacgtatattgaagggctgctgaaagtggtgcaccccgtgacgggcaaagtgcacacgatgttcaatcaggcgttgacgcaaaccgggcgcctcagctccgtcgaaccgaatttgcaaaacattccgattcggcttgaggaagggcggaaaatccgccaggcgttcgtgccgtcggagccggactggctcatctttgcggccgactattcgcaaatcgagctgcgcgtcctcgcccatatcgcggaagatgacaatttgattgaagcgttccggcgctggttggacatccatacgaaaacagccatggacattttccatgtgagcgaagaagacgtgacagccaacatgcgccgccaagcgaaggccgtcaattttggcatcgtgtacggcattagtgattacggtctggcgcaaaacttgaacattacgcgcaaagaagcggctgaatttattgagcgatattttgccagttttccaggtgtaaagcaatatatggacaacattgtgcaagaagcgaaacaaaaagggtatgtgacgacgctgctgcatcggcgccgctatttgcccgatattacaagccgcaacttcaacgtccgcacgttcgccgagcggacggcgatgaacacaccgatccagggatccgctgccgacatcattaagaaagcgatgatcgatctaagcgtgagcgtgcgcgaagaacggctgcaggcgcgcctgttgctgcaaggtcatgacgaactcattttggaggcgccgaaagaggaaatcggacggctgtgccgcctcgttccggaagtgatggagcaagccgtgacacttcgcgtgccgctgaaagtcgattaccattacggtccgacgtggtacgacgccaaataa

SEQ ID NO: 6 - DNA, 2451 bp, Homo sapiens
atggatgaagaggaactaatacaactaataatagaaaaaactggcaaatctcgagaggaaatagaaaaaatggtggaagaaaaaattaaagcttttaacaatttaatatctcgtaggggggctttactattagtagcaaaaaaacttggtgttttgtataaaaacactccgaaagagaaaaaaattggcgaattagaaagctgggaatatgtaaaagtaaagggcaaaattctcaaatcttttggattaattagttattcgaaagggaaattccaacctattattttaggagacgaaaccggtactattaaagctattatttggaataccgataaagaattacctgaaaacactgtaatagaagctattgggaaaaccaaaattaataagaaaactggcaatttagaattacatatagacagttataaaattttagaaagcgatttagagataaaaccccaaaagcaagaatttgttgggatttgcatagttaaatatccaaaaaaacaaacccaaaaaggcacaatagtatcgaaagcaattttaactagcttagatagggaattgcctgtagtatatttcaacgattttgattgggaaataggccatatatataaagtatatggaaagcttaagaaaaacataaaaactggtaaaatagaatttttcgctgacaaagttgaggaagcaacattaaaagatctaaaagcttttaaaggagaggccgatggaagcggaggggtcgacttggccgacaaagcggccctcgtcgtggaggtggtgggcgacaactatcaccatgccccgattgtcgggatcgccttggccaacgaacgcgggcggtttttcctgcgcccggagacggccgtcgccgatccgaaatttctcgcttggcttggcgatgagacgaagaaaaaaacgatgtttgattcaaagcgggcggccgtcgcgctaaatgggaaaggaatcgaactggctggcgtcggcgtcgtgttcgatctgttgctggccgcttacttgctcgatccggcgcaggcggcgggcgacgttgccgcggtggcgaaaatgcatcagtacgaggcggtgcgatcggatgaggcggtctatggaaaaggagcgaagcggacggttcctgatgaaccgacgcttgccgagcagctcgtccgcaaggcggcggccatttgggcgcttgaagagccgttgatggacgaactgcgccgcaacgaacaagatcggctgctgaccgagctcgaacacgcgctggctggcattttggccaatatggaatttactggagtgaaagtggacacgaagcggcttgaacagatgggggcggagctcaccgagcagctgcaggcggtcgagcggcgcatttacgaactcgccggccaagagttcaacattaactcgccgaaacagctcgggacggttttatttgacaagctgcagctcccggtgttgaaaaagacaaaaaccggctattcgacttcagccgatgtgctagaaaagcttgcaccgcaccatgaaatcgtcgaacatattttgcattaccgccaactcggcaagctgcagtcaacgtatattgaagggctgctgaaagtggtgcaccccgtgacgggcaaagtgcacacgatgttcaatcaggcgttgacgcaaaccgggcgcctcagctccgtcgaaccgaatttgcaaaacattccgattcggcttgaggaagggcggaaaatccgccaggcgttcgtgccgtcggagccggactggctcatctttgcggccgactattcgcaaatcgagctgcgcgtcctcgcccatatcgcggaagatgacaatttgattgaagcgttccggcgctggttggacatccatacgaaaacagccatggacattttccatgtgagcgaagaagacgtgacagccaacatgcgccgccaagcgaaggccgtcaattttggcatcgtgtacggcattagtgattacggtctggcgcaaaacttgaacattacgcgcaaagaagcggctgaatttattgagcgatattttgccagttttccaggtgtaaagcaatatatggacaacattgtgcaagaagcgaaacaaaaagggtatgtgacgacgctgctgcatcggcgccgctatttgcccgatattacaagccgcaacttcaacgtccgcacgttcgccgagcggacggcgatgaacacaccgatccagggatccgctgccgacatcattaagaaagcgatgatcgatctaagcgtgagcgtgcgcgaagaacggctgcaggcgcgcctgttgctgcaaggtcatgacgaactcattttggaggcgccgaaagaggaaatcggacggctgtgccgcctcgttccggaagtgatggagcaagccgtgacacttcgcgtgccgctgaaagtcgattaccattacggtccgacgtggtacgacgccaaataa

SEQ ID NO: 7 - DNA, 1992 bp, Homo sapiens
atggatgaagaggaactaatacaactaataatagaaaaaactggcaaatctcgagaggaaatagaaaaaatggtggaagaaaaaattaaagcttttaacaatttaatatctcgtaggggggctttactattagtagcaaaaaaacttggtgttttgtataaaaacactccgaaagagaaaaaaattggcgaattagaaagctgggaatatgtaaaagtaaagggcaaaattctcaaatcttttggattaattagttattcgaaagggaaattccaacctattattttaggagacgaaaccggtactattaaagctattatttggaataccgataaagaattacctgaaaacactgtaatagaagctattgggaaaaccaaaattaataagaaaactggcaatttagaattacatatagacagttataaaattttagaaagcgatttagagataaaaccccaaaagcaagaatttgttgggatttgcatagttaaatatccaaaaaaacaaacccaaaaaggcacaatagtatcgaaagcaattttaactagcttagatagggaattgcctgtagtatatttcaacgattttgattgggaaataggccatatatataaagtatatggaaagcttaagaaaaacataaaaactggtaaaatagaatttttcgctgacaaagttgaggaagcaacattaaaagatctaaaagcttttaaaggagaggccgatggaagcggaggggtcgacttggaactgcgccgcaacgaacaagatcggctgctgaccgagctcgaacacgcgctggctggcattttggccaatatggaatttactggagtgaaagtggacacgaagcggcttgaacagatgggggcggagctcaccgagcagctgcaggcggtcgagcggcgcatttacgaactcgccggccaagagttcaacattaactcgccgaaacagctcgggacggttttatttgacaagctgcagctcccggtgttgaaaaagacaaaaaccggctattcgacttcagccgatgtgctagaaaagcttgcaccgcaccatgaaatcgtcgaacatattttgcattaccgccaactcggcaagctgcagtcaacgtatattgaagggctgctgaaagtggtgcaccccgtgacgggcaaagtgcacacgatgttcaatcaggcgttgacgcaaaccgggcgcctcagctccgtcgaaccgaatttgcaaaacattccgattcggcttgaggaagggcggaaaatccgccaggcgttcgtgccgtcggagccggactggctcatctttgcggccgactattcgcaaatcgagctgcgcgtcctcgcccatatcgcggaagatgacaatttgattgaagcgttccggcgctggttggacatccatacgaaaacagccatggacattttccatgtgagcgaagaagacgtgacagccaacatgcgccgccaagcgaaggccgtcaattttggcatcgtgtacggcattagtgattacggtctggcgcaaaacttgaacattacgcgcaaagaagcggctgaatttattgagcgatattttgccagttttccaggtgtaaagcaatatatggacaacattgtgcaagaagcgaaacaaaaagggtatgtgacgacgctgctgcatcggcgccgctatttgcccgatattacaagccgcaacttcaacgtccgcacgttcgccgagcggacggcgatgaacacaccgatccagggatccgctgccgacatcattaagaaagcgatgatcgatctaagcgtgagcgtgcgcgaagaacggctgcaggcgcgcctgttgctgcaaggtcatgacgaactcattttggaggcgccgaaagaggaaatcggacggctgtgccgcctcgttccggaagtgatggagcaagccgtgacacttcgcgtgccgctgaaagtcgattaccattacggtccgacgtggtacgacgccaaataa

(19) United States
(12) Patent Application Publication
(10) Pub. No.: US 2021/0254034 A1
(43) Pub. Date: Aug. 19, 2021

(54) FUSION SINGLE-STRANDED DNA POLYMERASE BST, NUCLEIC ACID MOLECULE ENCODING FUSION DNA POLYMERASE NEQSSB-BST, METHOD OF PREPARATION AND UTILISATION THEREOF

(71) Applicant: INSTYTUT BIOTECHNOLOGII I MEDYCYNY MOLEKULARNEJ, Gdansk (PL)

(72) Inventors: Marta SPIBIDA, Kowal (PL); Kasjan SZEMIAKO, Gdansk (PL); Marcin OLSZEWSKI, Suwalki (PL); Dawid NIDZWORSKI, Gdansk (PL)

(73) Assignee: INSTYTUT BIOTECHNOLOGII I MEDYCYNY MOLEKULARNEJ, Gdansk (PL)

(21) Appl. No.: 17/253,445

(22) PCT Filed: Jun. 26, 2019
(86) PCT No.: PCT/PL2019/000046

§ 371 Date: Dec. 17, 2020

(30) Foreign Application Priority Data
Jun. 27, 2018 (PL) P.426093

(51) Int. Cl. C12N 9/12; C12N 15/62; C07K 14/195; C07K 1/36; C07K 1/22; C07K 1/26

(52) U.S. Cl. CPC C12N 9/1252; C12N 15/62; C07K 14/195; C07K 2319/80; C07K 1/22; C07K 1/26; C12Y 207/07007; C07K 1/36

(57) ABSTRACT

The subject of the invention is the fusion single-stranded DNA polymerase Bst linked with NeqSSB protein at the N-end of the polymerase using the linker consisting of six amino acids with the amino acid sequence Gly-Ser-Gly-Gly-Val-Asp, wherein the given polymerase is present in three different variants, and the preparation method thereof. Moreover, the subject of the invention is the nucleic acid molecule encoding the fusion DNA polymerase NeqSSB-Bst Full Length, Large Fragment, Short Fragment and their utilisation.

The subject of the present invention are the fusion single-stranded DNA polymerases Bst and the method of preparation thereof. The subject of the invention is also the nucleic acid molecule encoding the fusion polymerase NeqSSB-Bst according to the one of three variants of Bst polymerase: Full Length, Large Fragment, Short Fragment, and utilisation of the fusion DNA polymerases for isothermal amplification reactions.

DNA polymerases are the enzymes which play an essential role in the process of DNA replication and repair. They are widely used in various fields of science and are successfully utilised in sequencing or various PCR (Polymerase Chain Reaction) variants, where they catalyse the DNA synthesis processes in vitro, and this reaction is conducted in cycles with precisely defined thermal stages. Another and increasingly popular approach is the utilisation of the DNA polymerases in the isothermal techniques of DNA amplification which are not based on thermocycles and the reaction is conducted under constant temperature of elongation. Up to date, many such techniques have been developed for both DNA amplification and RNA amplification. The selection of the appropriate polymerase for a given technique depends mainly on its properties. Besides the basic polymerisation capabilities, the polymerases can also show the ability to hydrolyse DNA molecules thanks to the presence of the exonucleolytic domain or the reverse transcriptase activity. These characteristics are determined by the presence of the respective domains. The basic domains present in these enzymes are the polymerisation domain and 3′-5′ and 5′-3′ exonucleolytic domain. There are polymerases present where the deletion of the exonucleolytic domain leads to the obtaining of a functional protein with partially altered characteristics comparing with the native enzyme. The most popular polymerase of this type is Taq polymerase isolated from Thermus aquaticus bacterium, the discovery of which diametrically transformed the molecular biology. Without the 5′-3′ exonuclease activity the Taq [symbol] 1289 polymerase displays the increased thermostability, while it requires more Mg²⁺ ions and newly formed DNA strand contains fewer errors. The Bst polymerase is used for the isothermal amplification techniques. Its native form contains the non-active 3′-5′ exonucleolytic domain and active 5′-3′ exonucleolytic domain which activity can be disabled with a point mutation in position 73 (Tyr⁷³→Phe⁷³ and Tyr⁷³→Ala⁷³). This polymerase as well as Taq polymerase is the part of family A and is isolated from bacterium Bacillus stearothermophilus. Its optimum activity is about 60° C. and without the exonuclease activity the polymerase exhibits the strand displacement activity which is highly useful in Loop Mediated Isothermal Amplification (LAMP) reaction. The polymerase has an increased tolerance to clinical or environmental inhibitors comparing with other polymerases of this family, but still, taking into account the applications of this polymerase, it is important to seek the solutions which would lead mainly to the improvement of its processivity and the resistance to inhibitors.

NeqSSB protein is the member of Single-Stranded DNA Binding (SSB) protein family. The SSB proteins have various amino acid sequences and structures. However, they still contain one characteristic highly conserved Oligonucleotide/Oligosaccharide Binding (OB) Fold Domain consisting of about 100 amino acids. This domain is widely present in proteins exhibiting ssDNA-binding capability, and thus determines its basic characteristic common for all SSB proteins—non-specific binding of single-stranded DNA and, discovered far later, RNA binding capability. The SSB proteins play a key role in the processes closely associated with ssDNA. They are crucial in replication, recombination, and DNA repair. These proteins are responsible for the interactions with the single-stranded DNA, prevent the generation of secondary structures and protect against the degradation by nucleases.

The discovery of SSB proteins is dated for the first half of 1960s. The first SSB protein to be discovered are SSB proteins of T4 phage and E. coli. During the discovery, their high capabilities to interact with ssDNA interaction and to elute the protein using ssDNA-cellulose beads with high salt concentration (2 M NaCl) were elucidated. Additionally, the very high selectivity of this protein for the single-stranded DNA was also found out. The confirmation of the fundamental role of SSB proteins in the processes related to ssDNA is the fact that these proteins are present in all living organisms as well as the viruses.

The binding of SSB proteins with ssDNA is based on the packing of the aromatic amino acid residues between the residues of the oligonucleotide chain. Moreover, the positively-charged amino acid residues interact with the phosphate backbone of the ssDNA molecule.

Despite the fact that the NeqSSB protein belongs to the SSB family of proteins, it deviates with its characteristics from classical SSB proteins, hence it is referred to as NeqSSB-like protein. The protein originates from the hyperthermophilic archaeon Nanoarchaeum equitans, the parasite of craenarchaeon Ignicoccus hospitalis. The optimum growth conditions for this microorganisms require strictly anaerobic conditions and temperature of 90° C. Interestingly, Nanoarchaeum equitans contains the smallest known genome consisting of 490,885 base pairs. In contrast with most of known organisms with reduced genomes, this microorganism contains full set of enzymes taking part in replication, repair, and DNA recombination, including SSB protein.

The NeqSSB protein, as well as other proteins of this family, has the natural ability to bind DNA. It consists of 243 amino acid residues, and contains one OB domain in its structure and is biologically active as a monomer, similarly as in case of some viral SSB proteins. The reports show that comparing with other SSB proteins NeqSSB protein exhibits unusual capabilities concerning binding of all DNA forms (ssDNA, dsDNA), and mRNA without structural preferences. Moreover, the protein shows high thermostability. The half-life while maintaining the biological activity is 5 min in 100° C., while the melting temperature is 100.2° C.

To meet the requirements posed by modern diagnostics, molecular biology, or genetic engineering, it is necessary to improve the DNA polymerases to provide useful features in these fields of science. The modification introduced so far focus mainly on the introduction of the improved buffers, amplification reaction enhancers, or mutation of the DNA polymerases. The mutations lead to the obtaining of the enzymes with the increased thermostability and resistance to the inhibitors present in the clinical or environmental samples.

The DNA polymerase mechanism of action includes several significant steps. The first one consists of the attachment of the enzyme to the DNA matrix. Obtained DNA-DNA complex associates the respective dNTPs (deoxyribonucleotide triphosphates) as the result of the nucleophilic attack of the 3′ OH end on the nucleotide phosphorus atom. The last step leads to the generation of the phosphodiester bond and the release of the pyrophosphate.

One of the important stages of the polymerising actions of these enzymes, which is responsible for their final efficiency, is the initiation process related with the biding to the matrix DNA. Due to that reason the modification of the known polymerases is justified to facilitate the binding to the DNA strand subjected to polymerisation. An example of such an modification can be the generation of fusion DNA polymerases with proteins which exhibit a natural ability to bind single- and/or double-stranded DNA. The literature presents only several examples of such fusion DNA polymerases with the majority of them being fusions with thermostable enzymes used mainly for the Polymerase Chain Reactions.

The studies suggest that the fusion of the Taq, Pfu, Tpa, or KOD DNA polymerase with the DNA-binding protein Sso7d from hyperthermophilic archaeon Sulfolobus solfataricus, lead to the 5 to 17 times increase in the polymerase processivity. Similarly, the increase in the processivity and fidelity of the DNA polymerase of R869 bacteriophage was observed after fusion with its indigenous SSB protein (R869SSB) which binds single-stranded DNA.

The European Patent EP 1 934 372 B1 discloses the DNA polymerase of Thermococcus zilligi fused with SsoSSB protein of archaeon Sulfolobus solfataricus indicating the increase in the efficiency and the processivity of the modified enzyme.

Additionally, the fusion of TaqStoffel polymerase with NeqSSB protein capable of binding to the all types of DNA and with DBD domain of P. furiosus ligase was recently reported. Both fusions lead to the improvement in the functional features of enzymes, especially improved the processivity and thermostability of the native enzyme and significantly increased its tolerance to the clinical inhibitors (lactoferrin, heparin, whole blood). Small number of fusions of polymerases such as Bst and [symbol] 129 used in the isothermal reactions was also conducted. They are connected via HhH (Helix-hairpin-helix) domain of the topoisomerase V of Methanopyrus kandleri, what increased the affinity of the polymerase to DNA without a negative influence on the strand displacement activity (in fusion polymerases Bst and [symbol] 29). Moreover, higher fidelity and amplification efficiency using plasmid and genome DNA was observed (in case of [symbol] 129).

The literature presents also the fusion of Bst-like polymerase isolated from Geobacillus sp. 777. The chimers of the polymerase with DBD domain of the ligase Pyrococcus abyssi and Sto7d protein were generated and exhibited an increase in the processivity and the resistance to inhibitors (urea, whole blood, heparin, EDTA, NaCl, and ethanol) in comparison with the native polymerase.

The purpose of the present invention is to provide a fusion DNA polymerase Bst with NeqSSB protein binding to all types of DNA and RNA. Surprisingly, the problem was solved to a high extent in the present invention.

The subject of the present invention is a fusion DNA polymerase Bst with NeqSSB protein binding all types of DNA and RNA. Three Bst polymerase variants were subjected to the modifications: Full length—whole amino acid sequence of DNA|Bst polymerase with the disabled 5′-3′ activity thanks to the point mutation; Large Fragment—DNA|Bst polymerase without 5′-3′ domain and Short Fragment—short version with the deletion of both exonucleolytic domains. All variants of the Bst polymerase were fused with the NeqSSB protein by the polymerase N-end using a linker consisting of six amino acids.

The essence of the invention is the fusion polymerase of single-stranded DNA polymerase Bst or another polymerase of this class of DNA polymerases connected with NeqSSB protein or a protein with a sequence similar to NeqSSB in the degree not higher than 50% at N-end of the polymerase using a linker of an exemplary amino acid sequence Gly-Ser-Gly-Gly-Val-Asp or fused directly without a linker, wherein the mentioned polymerase is present in three different variants.

Fusion DNA polymerase NeqSSB-Bst containing one of the three Bst polymerase variants:
Full length—whole amino acid sequence of DNA polymerase|Bst with disabled 5′-3′ activity thanks to the point mutation;

Large Fragment—DNA polymerase|Bst without 5′-3′ domain;

Short Fragment—short version with the deletion of both exonucleolytic domains

Fusion DNA polymerase NeqSSB-Bst which binds to all types of DNA and RNA.
Fusion DNA polymerase NeqSSB-Bst with the sequence presented in SEQ1.
Fusion DNA polymerase NeqSSB-Bst with the sequence presented in SEQ2.
Fusion DNA polymerase NeqSSB-Bst with the sequence presented in SEQ3.
Nucleic acid molecule encoding the fusion DNA polymerase NeqSSB-Bst Full Length presented in SEQ 4.
Nucleic acid molecule encoding the fusion DNA polymerase NeqSSB-Bst Large Fragment presented in SEQ 5.
Nucleic acid molecule encoding the fusion DNA polymerase NeqSSB-Bst Short Fragment presented in SEQ 6.
Nucleic acid molecule encoding the fusion DNA polymerase NeqSSB-Bst defined above.
The preparation method of fusion DNA polymerase NeqSSB-Bst defined above, where:
first step includes the expression of the gene encoding the enzyme in the optimised conditions in microbiological shaker: growth temperature 28-37° C., incubation time of the medium after induction—3-20 h, inductor concentration—0.1-1 mM IPTG,
obtained cell lysate is subjected to the disintegration using ultrasound and elimination of the DNA genomic contamination using dsDNase.
second purification step utilises the metal affinity chromatography with His-Trap beads,
next steps cover triple dialysis (10 mM Tris-HCl pH 7.1, 50 mM KCl, 1 mM DTT, 0.1 mM EDTA, 50% Glycerol, 0.1% Triton X-100), gel filtration, and concentration of the preparation.
all processes were conducted in 4° C.,
the purity of the obtained proteins was tested using SDS-PAGE electrophoresis, and the number of units for the obtained preparation was determined using EvaEZ Fluorometric Polymerase Activity Assay Kit.

in vitro utilisation of the fusion single-stranded DNA polymerase Bst defined above for the isothermal amplification reactions.

### DESCRIPTION OF THE SEQUENCES AND FIGURES

Seq. 1—presents the amino acid sequence of the fusion polymerase NeqSSB-Bst Full length

Seq. 2—presents the amino acid sequence of the fusion polymerase NeqSSB-Bst Large Fragment

Seq. 3—presents the amino acid sequence of the fusion polymerase NeqSSB-Bst Short Fragment

Seq. 4—presents the sequence of the gene encoding fusion DNA polymerase NeqSSB-Bst Full Length

Seq. 5—presents the sequence of the gene encoding fusion DNA polymerase NeqSSB-Bst Large Fragment

Seq. 6—presents the sequence of the gene encoding fusion DNA polymerase NeqSSB-Bst Short Fragment

FIG. 1—presents the electrophoretic 10% polyacrylamide gel separation of proteins from individual stages of the fusion DNA polymerase purification
M—Protein mass marker (Thermo-Fischer Scientific) with the masses of the standard proteins: 116; 66.2; 45; 35; 25; 18.4; 14.4 kDa;
1—whole cell-free extract of the recombinant Escherichia coli TOP10F′-pETNeqSSB-Bst strain;
2—whole cell-free extract subjected to the preliminary thermal denaturation
3—fraction not bound with His-Trap column;
4—wash fraction of His-Trap beads containing 40 mM imidazole
5—wash fraction of His-Trap beads containing 100 mM imidazole
6—collected fraction containing fusion DNA polymerase after elution using 500 mM imidazole

FIG. 2—presents the charts concerning dependence of the EvaGreen dye fluorescence on time starting from the DNA amplification for the fusion DNA polymerases which enables the calculation of the number DNA polymerase units. The legend assigns the amounts of used DNA polymerase for the reaction in microliters to the curves.

FIG. 3—presents the electrophoretic 10% polyacrylamide gel separation of lysates for various expression conditions
M—Protein mass marker (Thermo-Fischer Scientific) with the masses of the standard proteins: 116; 66.2; 45; 35; 25; 18.4; 14.4 kDa;
1—whole cell-free extract of the recombinant Escherichia coli TOP10F′-pETNeqSSB-Bst strain before induction;
2—whole cell-free extract 3 h after induction with 1 mM IPTG, expression conducted in 28° C.;
3—whole cell-free extract 4 h after induction with 1 mM IPTG, expression conducted in 28° C.
4—whole cell-free extract 5 h after induction with 1 mM IPTG, expression conducted in 28° C.
5—whole cell-free extract 6 h after induction with 1 mM IPTG, expression conducted in 28° C.
6—whole cell-free extract 20 h after induction with 1 mM IPTG, expression conducted in 28° C.
7—whole cell-free extract 3 h after induction with 0.1 mM IPTG, expression conducted in 28° C.;
8—whole cell-free extract 4 h after induction with 0.1 mM IPTG, expression conducted in 28° C.
9—whole cell-free extract 5 h after induction with 0.1 mM IPTG, expression conducted in 28° C.
10—whole cell-free extract 6 h after induction with 0.1 mM IPTG, expression conducted in 28° C.
11—whole cell-free extract 20 h after induction with 0.1 mM IPTG, expression conducted in 28° C.
12—whole cell-free extract of the recombinant Escherichia coli TOP10F′-pETNeqSSB-Bst strain before induction;
13—whole cell-free extract 3 h after induction with 1 mM IPTG, expression conducted in 37° C.;
14—whole cell-free extract 4 h after induction with 1 mM IPTG, expression conducted in 37° C.
15—whole cell-free extract 5 h after induction with 1 mM IPTG, expression conducted in 37° C.
16—whole cell-free extract 6 h after induction with 1 mM IPTG, expression conducted in 37° C.
17—whole cell-free extract 20 h after induction with 1 mM IPTG, expression conducted in 37° C.
18—whole cell-free extract of the recombinant Escherichia coli TOP10F′-pETNeqSSB-Bst strain before induction;
19—whole cell-free extract 3 h after induction with 0.1 mM IPTG, expression conducted in 37° C.;
20—whole cell-free extract 4 h after induction with 0.1 mM IPTG, expression conducted in 37° C.
21—whole cell-free extract 5 h after induction with 0.1 mM IPTG, expression conducted in 37° C.
22—whole cell-free extract 6 h after induction with 0.1 mM IPTG, expression conducted in 37° C.
23—whole cell-free extract 20 h after induction with 0.1 mM IPTG, expression conducted in 37° C.

FIG. 4—shows the graphs presenting the change in the activity of the fusion DNA polymerases with the increase in temperature comparing with the reference DNA polymerase|Bst. The blue line presents the result of DNA polymerase|Bst, the red line for fusion DNA polymerase Bst Full length, violet line for fusion DNA polymerase Bst Large Fragment, and the green line for the fusion DNA polymerase Bst Short Fragment. The activity is described using the GelAnalyzer program based on the intensity of the obtained PCR products in an agarose gel.

FIG. 5—shows the electrophoretic separation in 1.5% agarose gel with ethidium bromide presenting the comparison of the processivity of DNA polymerases defined as the amplification rate during isothermal PCR. The reactions were conducted in various periods of time what is indicated over the lines.

FIG. 6—shows the electrophoretic separation in 1.5% agarose gel presenting the comparison of the DNA polymerase resistance to the inhibitors: blood lactoferrin (A), soil polyphenols (B).

### A:

A: 1—the reaction product generated as the result of the DNA amplification with added 6 μg of lactoferrin

2—the reaction product generated as the result of the DNA amplification with added 0.6 μg of lactoferrin

3—the reaction product generated as the result of the DNA amplification with added 0.06 μg of lactoferrin

4—the reaction product generated as the result of the DNA amplification with added 6 ng of lactoferrin

K+ reaction product generated during DNA amplification without the addition of an inhibitor.

### B:

1—the reaction product generated as the result of the DNA amplification with added 100 μg of polyphenols

2—the reaction product generated as the result of the DNA amplification with added 10 μg of polyphenols

3—the reaction product generated as the result of the DNA amplification with added 1 μg of polyphenols

4—the reaction product generated as the result of the DNA amplification with added 0.1 μg of polyphenols

5—the reaction product generated as the result of the DNA amplification with added 0.01 μg of polyphenols

K+ reaction product generated during DNA amplification without the addition of an inhibitor.

FIG. 7—shows the electrophoretic separation in 2% agarose gel with ethidium bromide presenting the results of the DNA electrophoretic mobility shift assay in the presence of the fusion DNA polymerases. The reaction mixture contained 10 pmol (dT₇₆) of fluorescein-labelled (green) and 2.5 pmol PCR product of 100 bp (orange)

1—d(T)₇₆
2—100 bp
3—d(T)₇₆+100 bp+3.3 pmol of the fusion DNA polymerase
4—d(T)₇₆+100 bp+6.6 pmol of the fusion DNA polymerase
5—d(T)₇₆+100 bp+13.2 pmol of the fusion DNA polymerase
6—d(T)₇₆+100 bp+26.4 pmol of the fusion DNA polymerase
7—d(T)₇₆+100 bp+52.8 pmol of the fusion DNA polymerase
8—d(T)₇₆+100 bp+105.6 pmol of the fusion DNA polymerase
9—d(T)₇₆+100 bp+211.2 pmol of the fusion DNA polymerase

The invention is illustrated by the embodiment, including but not limited to.

### EXAMPLE

### Fusion DNA Polymerase NeqSSB-Bst

Fusion DNA polymerases NeqSSB-Bst were obtained by fusion of three various Bst polymerases with NeqSSB protein at polymerase N-end using the linker consisting of six amino acids of the following sequence: Gly-Ser-Gly-Gly-Val-Asp. The sequences of the three variants of fusion DNA polymerase are presented in the figure SEQ. 1-3 (amino acid sequences) and SEQ. 4-6 (nucleotide sequences). DNA polymerases were obtained in the laboratory scale in the prokaryotic system based on Escherichia coli bacteria.

### Preparation—Example 1

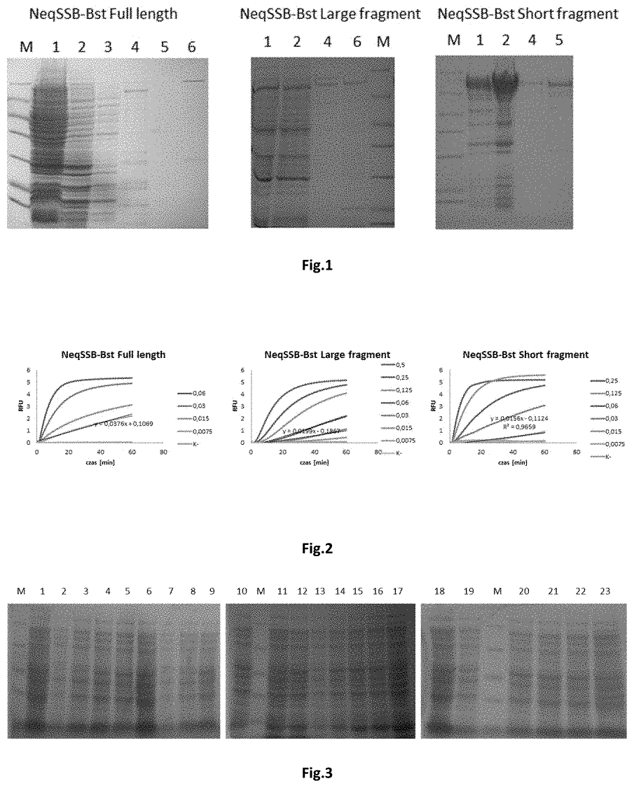

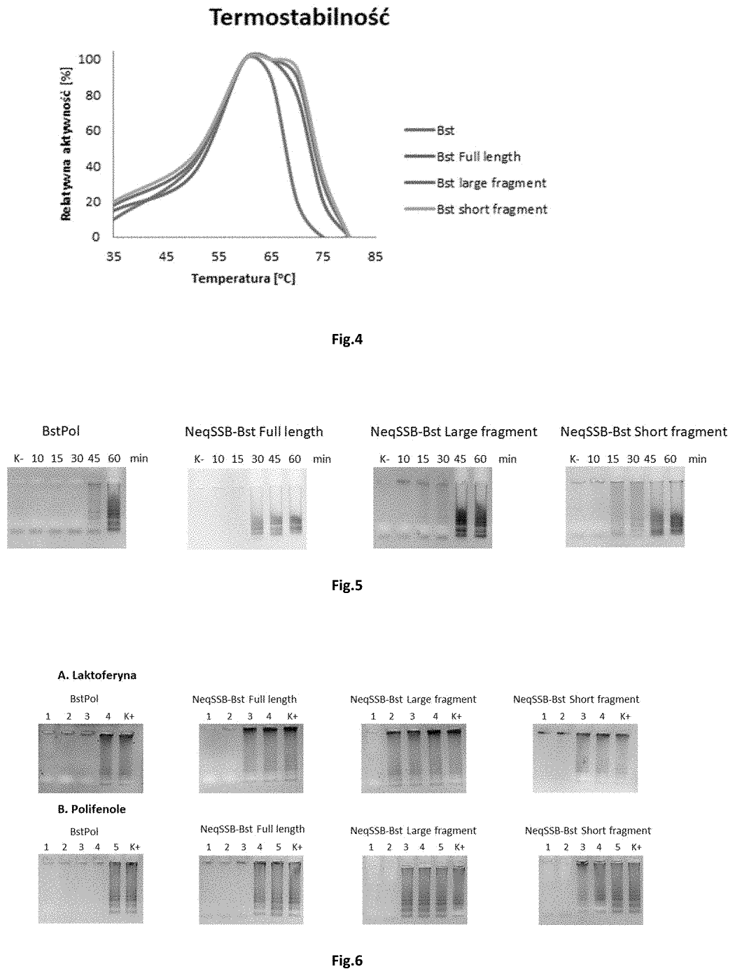

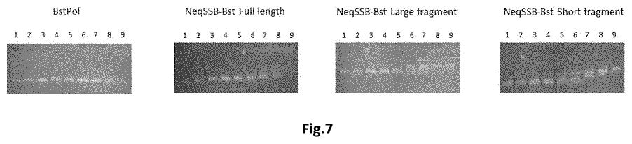

First step of the DNA polymerase preparation includes the expression of the gene encoding the enzyme in the optimised conditions in microbiological shaker: growth temperature—30° C., incubation time of the medium after induction—3 to 20 h, inductor concentration—0.1 to 1 mM IPTG. In the protein purification process the obtained cell lysate is subjected to the disintegration using ultrasound and removal of the DNA genomic contamination using dsDNase. Thanks to the presence of the oligohistidine domain, the second purification step utilises the metal affinity chromatography with His-Trap beads (FIG. 1). The next steps cover triple dialysis until obtaining the conditions which provide the stability for DNA polymerase (10 mM Tris-HCl pH 7.1, 50 mM KCl, 1 mM DTT, 0.1 mM EDTA, 50% Glycerol, 0.1% Triton X-100), gel filtration, and densification of the preparation. All the processes were conducted in 4° C. The purity of the obtained proteins was tested using SDS-PAGE electrophoresis, and the number of units for the obtained preparation was determined using EvaEZ Fluorometric Polymerase Activity Assay Kit Biotium (USA) according to the definition of unit: 1 activity unit [1 U] is the amount of DNA polymerase which can incorporate 10 nmol of nucleotides in 30 min in its optimum operation temperature 65° C. (FIG. 2). 1 litre of the laboratory scale culture provides approx. 5 mg of purified preparation with the activity of approx. 10 000 U, what enables the respective number of amplification reactions.

### Preparation—Example 2

Expression of the gene encoding the fusion DNA polymerase was conducted in the conditions providing appropriate oxygenation of the liquid culture in the temperature of 28° C. Logarithmic-phase cultures were induced using IPTG with the amount providing the protein expression—IPTG in the range of 1 mM to 0.1 mM and incubation of 3 to 20 hours (FIG. 3). After that the cell lysate was mechanically disintegrated and purified using metal affinity chromatography and ion-exchange chromatography. The obtained fusion DNA polymerases were subjected to the dialysis for the storage conditions (10 mM Tris-HCl pH 7.1, 50 mM KCl, 1 mM DTT, 0.1 mM EDTA, 50% Glycerol, 0.1% Triton X-100) and provided in the concentration of 1 U/μL based on the commercial EvaEZ Fluorometric Polymerase Activity Assay Kit of Biotium (USA) according to the definition of unit.

### Preparation—Example 3

An efficient expression of the gene encoding the polymerase Bst fused with NeqSSB protein was obtained in culture in 37° C. and induction of IPTG in range of 1 mM to 0.1 mM for 3 to 20 hours (FIG. 3). Centrifuged and mechanically disrupted cell lysate was purified using chromatographic techniques (metal affinity chromatography and ion-exchange chromatography), suspended in the formulation buffer (10 mM Tris-HCl pH 7.1, 50 mM KCl, 1 mM DTT, 0.1 mM EDTA, 50% Glycerol, 0.1% Triton X-100) and provided in the concentration of 1 U/μL. The amount of DNA units was defined based on the definition of unit using the EvaEZ Fluorometric Polymerase Activity Assay Kit of Biotium (USA).

The comparative analysis of the properties of the enzymes being the subject of the invention with the reference DNA polymerase Bst have shown that the presence of an additional DNA-binding NeqSSB protein has a positive impact on the DNA polymerase properties. The thermostability of the all obtained fusion variants of the DNA polymerases in comparison with the reference DNA polymerase Bst was increased by approx. 20% (FIG. 4). Moreover, the DNA polymerases in fusion with NeqSSB protein shown the threefold increase of processivity (FIG. 5). The fusion DNA polymerases tolerate the concentration of clinical (lactoferrin, heparin) and environmental (humic acid, soil, polyphenols) inhibitors in the reaction mixtures increased even by several dozen of times comparing with the reference polymerase (FIG. 6). The fusion DNA polymerases exhibited increased sensitivity by several times and thus increased affinity to the DNA matrix comparing with the reference DNA polymerase Bst.

### BIBLIOGRAPHY

[1] Patel P. H., Motoshi S., Adman E., Shinkai A, Prokaryotic DNA Polymerase Evolution, Structure and Base Flipping Mechanism for Nucleotide Selection, J Mol Biol., 2001, 308: 823-837.
[2] Steitz T. A., The Journal of Biological Chemistry, 1999, 274: 17395-17398
[3] Riggs M. G., Tudor S., Sivaram M., McDonough S. H. Construction of single amino acid substitution mutants of cloned Bacillus stearothermophilusDNA polymerase I which lack 5′→3′ exonuclease activity. Biochim Biophys Acta, 1996; 1307(2): 178-86.
[4] Oscorbin I. P., Belousova E. A., Boyarskikh U. A., Zakabunin A. I., Khrapov E. A., Filipenko M. L., Derivatives of Bst-like Gss-polymerase with improved processivity and inhibitor tolerance, Nucleic Acids Research, 2017, 45 (16): 9595-9610
[5] Phang S. M., Teo C. Y., Lo E., Wong V. W. Cloning and complete sequence of the DNA polymerase-encoding gene (Bstpoll) and characterisation of the Klenow-like fragment from Bacillus stearothermophilus. Gene. 1995; 163(1):65-8.
[6] Nowak M, Olszewski M, Śpibida M, Kur J. Characterization of single-stranded DNA-binding proteins from the psychrophilic bacteria Desulfotalea psychrophila, Flavobacterium psychrophilum, Psychrobacter arcticus, Psychrobacter cryohalolentis, Psychromonas ingrahamii, Psychroflexus torquis, and Photobacterium profundum. BMC Microbiol. 2014; 14:91.
[7] Kur J, Olszewski M, Długołȩcka A, Filipkowski P. Single-stranded DNA-binding proteins (SSBs)—sources and applications in molecular biology. Acta Biochim Pol. 2005; 52(3):569-74.
[8] Sigal N, Delius H, Kornberg T, Gefter M L, Alberts B. A DNA-unwinding protein isolated from Escherichia coli: its interaction with DNA and with DNA polymerases. Proc Natl Acad Sci USA. 1972; 69(12):3537-41.
[9] Olszewski M, Balsewicz J, Nowak M, Maciejewska N, Cyranka-Czaja A, Zalewska-Pia̧tek B, Pia̧tek R, Kur J. Characterization of a Single-Stranded DNA-Binding-Like Protein from Nanoarchaeum equitans—A Nucleic Acid Binding Protein with Broad Substrate Specificity. PLoS One. 2015; 10(5):e0126563
[10] Kim K. P., Cho S. S., Lee K. K., Youn M. H., Kwon S. T. Improved thermostability and PCR efficiency of Thermococcus celericrescens DNA polymerase via site-directed mutagenesis, J Biotechnol., 2011, 10; 155 (2): 156-63
[11] Kermekchiev M. B., Kirilova L. I., Vail E. E., Barnes W. M., Mutants of Taq DNA polymerase resistant to PCR inhibitors allow DNA amplification from whole blood and crude soil samples, Nucleic Acids Res, 2009, 37(5): e40
[12] Patel P H, Suzuki M, Adman E, Shinkai A, Loeb L A. Prokaryotic DNA polymerase I: evolution, structure, and “base flipping” mechanism for nucleotide selection. J Mol Biol. 2001; 308(5):823-37
[13] Wang Y., Prosen D. E., Mei L., Sullivan J. C., Finney M., Vander Horn P. B., A novel strategy to engineer DNA polymerases for enhanced processivity and improved performance In vitro, 2004 Nucleic Acid Research, 32 (3)
[14] Lee J. I., Cho S. S., Eui-JoonKil E. J., Kwon S. T., Characterization and PCR application of a thermostable DNA polymerase from Thermococcus pacificu, Enzyme and Microbial Technology 47 (2010) 147-152
[15] Wang F, Li S, Zhao H, Bian L, Chen L, Zhang Z, Zhong X, Ma L, Yu X. Expression and Characterization of the RKOD DNA Polymerase in Pichia pastoris. PLoS One. 2015; 10(7):e0131757.
[16] Sun S., Geng L., Shamoo Y., Structure and Enzymatic Properties of a Chimeric Bacteriophage R869 DNA Polymerase and Single-Stranded DNA Binding Protein with Increased processivity, PROTEINS: Structure, Function, and Bioinformatics, 2006, 65:231-238
[17] SSB—POLYMERASE FUSION PROTEINS, European Patent Office, EP 1934372 B1, date of filing: 8, Sep. 2006, date of publication: 20, Feb. 2013
[18] Śpibida M, Krawczyk B, Zalewska-Pia̧tek B, Pia̧tek R, Wysocka M, Olszewski M. Fusion of DNA-binding domain of Pyrococcus furiosus ligase with TaqStoffel DNA polymerase as a useful tool in PCR with difficult targets. Appl Microbiol Biotechnol. 2018; 102(2):713-721.
[19] Olszewski M, Spibida M, Bilek M, Krawczyk B. Fusion of Taq DNA polymerase with single-stranded DNA binding-like protein of Nanoarchaeum equitans-Expression and characterization. PLoS One. 2017; 12(9):e0184162
[20] de Vega M., Lázaro J. M., Mencía M., Blanco L., Salas M., Improvement of [symbol] 29 DNA polymerase amplification performance by fusion of DNA binding motifs, PANS, 2010; 107 (38):16506-16511
[21] Pavlov A. R., Pavlova N. V., Kozyavkin S. A., Slesarev A. I., Cooperation between Catalytic and DNA-binding Domains Enhances Thermostability and Supports DNA Synthesis at Higher Temperatures by Thermostable DNA Polymerases, Biochemistry. 2012; 51(10): 2032-2043.
[22] Tveit H., Kristensen T., Fluorescence-Based DNA Polymerase Assay. Anal Biochem. 2001; 289:96-8.

Nucleotide and amino acid sequence
SEQ. 1
MDEEELIQLIIEKTGKSREEIEKMVEEKIKAFNNLISRRGALLLVAKKLGVLYKNTPKEKKIGELESWEYVKVKG
KILKSFGLISYSKGKFQPIILGDETGTIKAIIWNTDKELPENTVIEAIGKTKINKKTGNLELHIDSYKILESDLE
IKPQKQEFVGICIVKYPKKQTQKGTIVSKAILTSLDRELPVVYFNDFDWEIGHIYKVYGKLKKNIKTGKIEFFAD
KVEEATLKDLKAFKGEADGSGGVDLKNKLVLIDGNSVAYRAFFALPLLHNDKGIHTNAVYGFTMMLNKILAEEQPT
HILVAFDAGKTTFRHETFQDAKGGRQQTPPELSEQFPLVRELLKAYRIPAYELDHYEADDIIGTMAARAEREG
FAVKVISGDRDLTQLASPQVIVEITKKGITDIESYTPETVVEKYGLIPEQIVDLKGLMGDKSDNIPGVPGIGKK
TAVKLLKQFGTVENVIASIDEIKGEKLKENLRQYRDLALLSKCILAAICRDAPVELTLDDIVYKGEDREKVVALF
QELGFQSFLDKMAVQTDEGEKPLAGMDFAIADSVTMEMLADKAALVVEVVGDNYHHAPIVGIALANERGR
FFLRPETAVADPKFLAWLGDETKKKTMFDSKRAAVALNGKGIELAGVGVVFDLLLAAYLLDPAQAAGDVAA
VAKMHQYEAVRSDEAVYGKGAKRTVPDEPTLAEQLVRKAAAIWALEEPLMDELRRNEQDRLLTELEHALA
GILANMEFTGVKVDTKRLEQMGAELTEQLQAVERRIYELAGQEFNINSPKQLGTVLFDKLQLPVLKKTKTGY
STSADVLEKLAPHHEIVEHILHYRQLGKLQSTYIEGLLKVVHPVTGKVHTMFNQALTQTGRLSSVEPNLQNIPI
RLEEGRKIRQAFVPSEPDWLIFAADYSQIELRVLAHIAEDDNLIEAFRRWLDIHTKTAMDIFHVSEEDVTANM
RRQAKAVNFGIVYGISDYGLAQNLNITRKEAAEFIERYFASFPGVKQYMDNIVQEAKQKGYVTILLHRRRYLP
DITSRNFNVRTFAERTAMNTPIQGSAADIIKKAMIDLSVSVREERLQARLLLQGHDELILEAPKEEIGRLCRLVP
EVMECIAVTLRVPLKVDYHYGPTVVYDAK.
LENGTH: 1127 aa
TYPE: amino acid sequence
TYPE OF THE MOLECULAR: Protein
SEQ. 2
MDEEELIQLIIEKTGKSREEIEKMVEEKIKAFNNLISRRGALLLVAKKLGVLYKNTPKEKKIGELESWEYVKVKG
KILKSFGLISYSKGKFQPIILGDETGTIKAIIWNTDKELPENTVIEAIGKTKINKKTGNLELHIDSYKILESDLE
IKPQKQEFVGICIVKYPKKQTQKGTIVSKAILTSLDRELPVVYFNDFDWEIGHIYKVYGKLKKNIKTGKIEFFAD
KVEEATLKDLKAFKGEADGSGGVDLADKAALVVEVVGDNYHHAPIVGIALANERGRFFLRPETAVADPKFLAWLG
DETKKKTMFDSKRAAVALNGKGIELAGVGVVFDLLLAAYLLDPAQAAGDVAAVAKMHQYEAVRSDEAVYG
KGAKRTVPDEPTLAEQLVRKAAAIWALEEPLMDELRRNEQDRLLTELEHALAGILANMEFTGVKVDTKRLE
QMGAELTEQLQAVERRIYELAGQEFNINSPKQLGTVLFDKLQLPVLKKTKTGYSTSADVLEKLAPHHEIVEHIL
HYRQLGKLQSTYIEGLLKVVHPVTGKVHTMFNQALTQTGRLSSVEPNLQNIPIRLEEGRKIRQAFVPSEPDW
LIFAADYSQIELRVLAHIAEDDNLIEAFRRWLDIHTKTAMDIFHVSEEDVTANMRRQAKAVNFGIVYGISDYG
LAQNLNITRKEAAEFIERYFASFPGVKQYMDNIVQEAKQKGYVTTLLHRRRYLPDITSRNFNVRTFAERTAM
NTPIQGSAADIIKKAMIDLSVSVREERLQARLLLQGHDELILEAPKEEIGRLCRLVPEVMEQAVTLRVPLKVDY
HYGPTVVYDAK.
LENGTH: 816 aa
TYPE: amino acid sequence
TYPE OF THE MOLECULAR: Protein
SEQ. 3
MDEEELIQLIIEKTGKSREEIEKMVEEKIKAFNNLISRRGALLLVAKKLGVLYKNTPKEKKIGELESWEYVKVKG
KILKSFGLISYSKGKFQPIILGDETGTIKAIIWNTDKELPENTVIEAIGKTKINKKTGNLELHIDSYKILESDLE
IKPQKQEFVGICIVKYPKKQTQKGTIVSKAILTSLDRELPVVYFNDFDWEIGHIYKVYGKLKKNIKTGKIEFFAD
KVEEATLKDLKAFKGEADGSGGVDLELRRNEQDRLLTELEHALAGILANMEFTGVKVDTKRLEQMGAELTEQLQA
VERRIYELAGQEFNINSPKOLGTVLFDKLQLPVLKKTKTGYSTSADVLEKLAPHHEIVEHILHYRQLGKLQSTY1
EGLLKVVHPVTGKVHTMFNQALTQTGRLSSVEPNLQNIPIRLEEGRKIRQAFVPSEPDWLIFAADYSQIELRV
LAHIAEDDNLIEAFRRWLDIHTKTAMDIFHVSEEDVTANMRRQAKAVNFGIVYGISDYGLAQNLNITRKEAA
EFIERYFASFPGVKQYMDNIVQEAKQKGYVTTLLHRRRYLPDITSRNFNVRTFAERTAMNTPIQGSAADIIKK
AMIDLSVSVREERLQARLLLQGHDELILEAPKEEIGRLCRLVPEVMEQAVTLRVPLKVDYHYGPTWYDAK.
LENGTH: 663 aa
TYPE: amino acid sequence
TYPE OF THE MOLECULAR: Protein
SEQ. 4
ATGGATGAAGAGGAACTAATACAACTAATAATAGAAAAAACTGGCAAATCTCGAGAGGAAATAGAAA
AAATGGTGGAAGAAAAAATTAAAGCTTTTAACAATTTAATATCTCGTAGGGGGGCTTTACTATTAGTAG
CAAAAAAACTTGGTGTTTTGTATAAAAACACTCCGAAAGAGAAAAAAATTGGCGAATTAGAAAGCTGG
GAATATGTAAAAGTAAAGGGCAAAATTCTCAAATCTTTTGGATTAATTAGTTATTCGAAAGGGAAATTC
CAACCTATTATTTTAGGAGACGAAACCGGTACTATTAAAGCTATTATTTGGAATACCGATAAAGAATTA
CCTGAAAACACTGTAATAGAAGCTATTGGGAAAACCAAAATTAATAAGAAAACTGGCAATTTAGAATTA
CATATAGACAGTTATAAAATTTTAGAAAGCGATTTAGAGATAAAACCCCAAAAGCAAGAATTTGTTGGG
ATTTGCATAGTTAAATATCCAAAAAAACAAACCCAAAAAGGCACAATAGTATCGAAAGCAATTTTAACT
AGCTTAGATAGGGAATTGCCTGTAGTATATTTCAACGATTTTGATTGGGAAATAGGCCATATATATAAA
GTATATGGAAAGCTTAAGAAAAACATAAAAACTGGTAAAATAGAATTTTTCGCTGACAAAGTTGAGGA
AGCAACATTAAAAGATCTAAAAGTTTAAAGGAGAGGCCGATGGAAGCGGAGGGGTCGACTTGAAA
AACAAGCTCGTCTTAATTGACGGCAACAGCGTGGCGTACCGCGCCTTTTTTGCGTTGCCGCTTTTGCATA
ACGATAAAGGGATTCATACGAACGCAGTCTACGGGTTTACGATGATGTTAAACAAAATTTTGGCGGAA
GAGCAGCCGACCCACATTCTCGTTGCGTTTGACGCCGGGAAAACGACGTTCCGCCATGAAACGTTCCAA
GACGCCAAAGGCGGGCGGCAGCAGACGCCGCCGGAACTGTCGGAACAGTTTCCGCTCGTGCGCGAAT
TGCTCAAAGCGTACCGCATCCCCGCCTATGAGCTCGACCATTATGAAGCGGATGACATCATCGGAACGA
TGGCGGCGCGGGCTGAGCGAGAAGGGTTTGCAGTGAAAGTCATTTCCGGCGACCGCGATTTAACCCA
GCTTGCTTCCCCGCAAGTGACGGTGGAGATTACGAAAAAAGGGATTACCGACATCGAGTCGTACACGC
CGGAGACGGTCGTGGAAAAATACGGCCTCACCCCGGAGCAAATTGTCGACTTGAAAGGATTGATGGG
CGACAAATCCGACAACATCCCTGGCGTGCCCGGCATCGGGAAAAAAACAGCCGTCAAGCTGCTCAAGC
AATTCGGCACGGTCGAAAACGTACTGGCATCGATCGATGAGATCAAAGGGGAGAAGCTGAAAGAAAA
TTTGCGCCAATACCGGGATTTGGCGCTTTTAAGCAAACAGCTGGCCGCTATTTGCCGCGACGCCCCGGT
TGAGCTGACGCTCGATGACATTGTCTACAAAGGAGAAGACCGGGAAAAAGTGGTCGCCTTGTTTCAGG
AGCTCGGATTCCAGTCGTTTCTCGACAAGATGGCCGTCCAAACGGATGAAGGCGAAAAGCCGCTCGCC
GGGATGGATTTTGCGATCGCCGACAGCGTCACGGACGAAATGCTCGCCGACAAAGCGGCCCTCGTCGT
GGAGGTGGTGGGCGACAACTATCACCATGCCCCGATTGTCGGGATCGCCTTGGCCAACGAACGCGGG
CGGTTTTTCCTGCGCCCGGAGACGGCCGTCGCCGATCCGAAATTTCTCGCTTGGCTTGGCGATGAGACG
AAGAAAAAAACGATGTTTGATTCAAAGCGGGCGGCCGTCGCGCTAAATGGGAAAGGAATCGAACTGG
CTGGCGTCGGCGTCGTGTTCGATCTGTTGCTGGCCGCTTACTTGCTCGATCCGGCGCAGGCGGCGGGC
GACGTTGCCG CGGTGGCGAAAATGCATCAGTACGAGGCGGTGCGATCGGATGAGGCGGTCTATGGAA
AAGGAGCGAAGCGGACGGTTCCTGATGAACCGACGCTTGCCGAGCAGCTCGTCCGCAAGGCGGCGGC
CATTTGGGCGCTTGAAGAGCCGTTGATGGACGAACTGCGCCGCAACGAACAAGATCGGCTGCTGACCG
AGCTCGAACACGCGCTGGCTGGCATTTTGGCCAATATGGAATTTACTGGAGTGAAAGTGGACACGAAG
CGGCTTGAACAGATGGGGGCGGAGCTCACCGAGCAGCTGCAGGCGGTCGAGCGGCGCATTTACGAAC
TCGCCGGCCAAGAGTTCAACATTAACTCGCCGAAACAGCTCGGGACGGTTTTATTTGACAAGCTGCAGC
TCCCGGTGTTGAAAAAGACAAAAACCGGCTATTCGACTTCAGCCGATGTGCTAGAAAAGCTTGCACCG
CACCATGAAATCGTCGAACATATTTTGCATTACCGCCAACTCGGCAAGCTGCAGTCAACGTATATTGAA
GGGCTGCTGAAAGTGGTGCACCCCGTGACGGGCAAAGTGCACACGATGTTCAATCAGGCGTTGACGC
AAACCGGGCGCCTCAGCTCCGTCGAACCGAATTTGCAAAACATTCCGATTCGGCTTGAGGAAGGGCGG
AAAATCCGCCAGGCGTTCGTGCCGTCGGAGCCGGACTGGCTCATCTTTGCGGCCGACTATTCGCAAATC
GAGCTGCGCGTCCTCGCCCATATCGCGGAAGATGACAATTTGATTGAAGCGTTCCGGCGCTGGTTGGA
CATCCATACGAAAACAGCCATGGACATTTTCCATGTGAGCGAAGAAGACGTGACAGCCAACATGCGCC
GCCAAGCGAAGGCCGTCAATTTTGGCATCGTGTACGGCATTAGTGATTACGGTCTGGCGCAAAACTTG
AACATTACGCGCAAAGAAGCGGCTGAATTTATTGAGCGATATTTTGCCAGTTTTCCAGGTGTAAAGCAA
TATATGGACAACATTGTGCAAGAAGCGAAACAAAAAGGGTATGTGACGACGCTGCTGCATCGGCGCC
GCTATTTGCCCGATATTACAAGCCGCAACTTCAACGTCCGCACGTTCGCCGAGCGGACGGCGATGAACA
CACCGATCCAGGGATCCGCTGCCGACATCATTAAGAAAGCGATGATCGATCTAAGCGTGAGCGTGCGC
GAAGAACGGCTGCAGGCGCGCCTGTTGCTGCAAGGTCATGACGAACTCATTTTGGAGGCGCCGAAAG
AGGAAATCGGACGGCTGTGCCGCCTCGTTCCGGAAGTGATGGAGCAAGCCGTGACACTTCGCGTGCCG
CTGAAAGTCGATTACCATTACGGTCCGACGTGGTACGACGCCAAATAA.
LENGTH: 3384 nucleotides
TYPE: nucleic acid
TOPOLOGY: plasmid
NUBER OF STRANDS: one
TYPE OF THE MOLECULAR: DNA
SEQ. 5
ATGGATGAAGAGGAACTAATACAACTAATAATAGAAAAAACTGGCAAATCTCGAGAGGAAATAGAAA
AAATGGTGGAAGAAAAAATTAAAGOTTTAACAATTTAATATCTCGTAGGGGGGCTTTACTATTAGTAG
CAAAAAAACTTGGTGTTTTGTATAAAAACACTCCGAAAGAGAAAAAAATTGGCGAATTAGAAAGCTGG
GAATATGTAAAAGTAAAGGGCAAAATTCTCAAATCTTTTGGATTAATTAGTTATTCGAAAGGGAAATTC
CAACCTATTATTTTAGGAGACGAAACCGGTACTATTAAAGCTATTATTTGGAATACCGATAAAGAATTA
CCTGAAAACACTGTAATAGAAGCTATTGGGAAAACCAAAATTAATAAGAAAACTGGCAATTTAGAATTA
CATATAGACAGTTATAAAATTTTAGAAAGCGATTTAGAGATAAAACCCCAAAAGCAAGAATTTGTTGGG
ATTTGCATAGTTAAATATCCAAAAAAACAAACCCAAAAAGGCACAATAGTATCGAAAGCAATTTTAACT
AGCTTAGATAGGGAATTGCCTGTAGTATATTTCAACGATTTTGATTGGGAAATAGGCCATATATATAAA
GTATATGGAAAGUTAAGAAAAACATAAAAACTGGTAAAATAGAATTTTTCGCTGACAAAGTTGAGGA
AGCAACATTAAAAGATCTAAAAGCTTTTAAAGGAGAGGCCGATGGAAGCGGAGGGGTCGACTTGGCC
GACAAAGCGGCCCTCGTCGTGGAGGTGGTGGGCGACAACTATCACCATGCCCCGATTGTCGGGATCGC
CTTGGCCAACGAACGCGGGCGGTTTTTCCTGCGCCCGGAGACGGCCGTCGCCGATCCGAAATTTCTCG
CTTGGCTTGGCGATGAGACGAAGAAAAAAACGATGTTTGATTCAAAGCGGGCGGCCGTCGCGCTAAAT
GGGAAAGGAATCGAACTGGCTGGCGTCGGCGTCGTGTTCGATCTGTTGCTGGCCGCTTACTTGCTCGA
TCCGGCGCAGGCGGCGGGCGACGTTGCCGCGGTGGCGAAAATGCATCAGTACGAGGCGGTGCGATCG
GATGAGGCGGTCTATGGAAAAGGAGCGAAGCGGACGGTTCCTGATGAACCGACGCTTGCCGAGCAGC
TCGTCCGCAAGGCGGCGGCCATTTGGGCGCTTGAAGAGCCGTTGATGGACGAACTGCGCCGCAACGA
ACAAGATCGGCTGCTGACCGAGCTCGAACACGCGCTGGCTGGCATTTTGGCCAATATGGAATTTACTG
GAGTGAAAGTGGACACGAAGCGGCTTGAACAGATGGGGGCGGAGCTCACCGAGCAGCTGCAGGCGG
TCGAGCGGCGCATTTACGAACTCGCCGGCCAAGAGTTCAACATTAACTCGCCGAAACAGCTCGGGACG
GTTTTATTTGACAAGCTGCAGCTCCCGGTGTTGAAAAAGACAAAAACCGGCTATTCGACTTCAGCCGAT
GTGCTAGAAAAGCTTGCACCGCACCATGAAATCGTCGAACATATTTTGCATTACCGCCAACTCGGCAAG
CTGCAGTCAACGTATATTGAAGGGCTGCTGAAAGTGGTGCACCCCGTGACGGGCAAAGTGCACACGAT
GTTCAATCAGGCGTTGACGCAAACCGGGCGCCTCAGCTCCGTCGAACCGAATTTGCAAAACATTCCGAT
TCGGCTTGAGGAAGGGCGGAAAATCCGCCAGGCGTT*CGTGCCGTCGGAGCCGGACTGGCTCATCTTTG
CGGCCGACTATTCGCAAATCGAGCTGCGCGTCCTCGCCCATATCGCGGAAGATGACAATTTGATTGAAG
CGTTCCGGCGCTGGTTGGACATCCATACGAAAACAGCCATGGACATTTTCCATGTGAGCGAAGAAGAC
GTGACAGCCAACATGCGCCGCCAAGCGAAGGCCGTCAATTTTGGCATCGTGTACGGCATTAGTGATTA
CGGTCTGGCGCAAAACTTGAACATTACGCGCAAAGAAGCGGCTGAATTTATTGAGCGATATTTTGCCA
GTTTTCCAGGTGTAAAGCAATATATGGACAACATTGTGCAAGAAGCGAAACAAAAAGGGTATGTGACG
ACGCTGCTGCATCGGCGCCGCTATTTGCCCGATATTACAAGCCGCAACTTCAACGTCCGCACGT1CGCC
GAGCGGACGGCGATGAACACACCGATCCAGGGATCCGCTGCCGACATCATTAAGAAAGCGATGATCG
ATCTAAGCGTGAGCGTGCGCGAAGAACGGCTGCAGGCGCGCCTGTTGCTGCAAGGTCATGACGAACTC
ATTTTGGAGGCGCCGAAAGAGGAAATCGGACGGCTGTGCCGCCTCGTTCCGGAAGTGATGGAGCAAG
CCGTGACACTTCGCGTGCCGCTGAAAGTCGATTACCATTACGGTCCGACGTGGTACGACGCCAAATAA.
LENGTH: 2451 nucleotides
TYPE: nucleic acid
TOPOLOGY: plasmid
NUMBER OF STRANDS: one
TYPE OF THE MOLECULAR: DNA
SEQ. 6
ATGGATGAAGAGGAACTAATACAACTAATAATAGAAAAAACTGGCAAATCTCGAGAGGAAATAGAAA
AAATGGTGGAAGAAAAAATTAAAGCTTTTAACAATTTAATATCTCGTAGGGGGGCTTTACTATTAGTAG
CAAAAAAACTTGGTGTTTTGTATAAAAACACTCCGAAAGAGAAAAAAATTGGCGAATTAGAAAGCTGG
GAATATGTAAAAGTAAAGGGCAAAATTCTCAAATCTTTTGGATTAATTAGTTATTCGAAAGGGAAATTC
CAACCTATTATTTTAGGAGACGAAACCGGTACTATTAAAGCTATTATTTGGAATACCGATAAAGAATTA
CCTGAAAACACTGTAATAGAAGCTATTGGGAAAACCAAAATTAATAAGAAAACTGGCAATTTAGAATTA
CATATAGACAGTTATAAAATTTTAGAAAGCGATTTAGAGATAAAACCCCAAAAGCAAGAATTTGTTGGG
ATTTGCATAGTTAAATATCCAAAAAAACAAACCCAAAAAGGCACAATAGTATCGAAAGCAATTTTAACT
AGCTTAGATAGGGAATTGCCTGTAGTATATTTCAACGATTTTGATTGGGAAATAGGCCATATATATAAA
GTATATGGAAAGCTTAAGAAAAACATAAAAACTGGTAAAATAGAATTTTTCGCTGACAAAGTTGAGGA
AGCAACATTAAAAGATCTAAAAGCTTTTAAAGGAGAGGCCGATGGAAGCGGAGGGGTCGACTTGGAA
CTGCGCCGCAACGAACAAGATCGGCTGCTGACCGAGCTCGAACACGCGCTGGCTGGCATTTTGGCCAA
TATGGAATTTACTGGAGTGAAAGTGGACACGAAGCGGCTTGAACAGATGGGGGCGGAGCTCACCGAG
CAGCTGCAGGCGGTCGAGCGGCGCATTTACGAACTCGCCGGCCAAGAGTTCAACATTAACTCGCCGAA
ACAGCTCGGGACGGTTTTATTTGACAAGCTGCAGCTCCCGGTGTTGAAAAAGACAAAAACCGGCTATTC
GACTTCAGCCGATGTGCTAGAAAAGCTTGCACCGCACCATGAAATCGTCGAACATATTTTGCATTACCG
CCAACTCGGCAAGCTGCAGTCAACGTATATTGAAGGGCTGCTGAAAGTGGTGCACCCCGTGACGGGCA
AAGTGCACACGATGTTCAATCAGGCGTTGACGCAAACCGGGCGCCTCAGCTCCGTCGAACCGAATTTG
CAAAACATTCCGATTCGGCTTGAGGAAGGGCGGAAAATCCGCCAGGCGTTCGTGCCGTCGGAGCCGG
ACTGGCTCATCTTTGCGGCCGACTATTCGCAAATCGAGCTGCGCGTCCTCGCCCATATCGCGGAAGATG
ACAATTTGATTGAAGCGTTCCGGCGCTGGTTGGACATCCATACGAAAACAGCCATGGACATTTTCCATG
TGAGCGAAGAAGACGTGACAGCCAACATGCGCCGCCAAGCGAAGGCCGTCAATTTTGGCATCGTGTAC
GGCATTAGTGATTACGGTCTGGCGCAAAACTTGAACATTACGCGCAAAGAAGCGGCTGAATTTATTGA
GCGATATTTTGCCAGTTTTCCAGGTGTAAAGCAATATATGGACAACATTGTGCAAGAAGCGAAACAAA
AAGGGTATGTGACGACGCTGCTGCATCGGCGCCGCTATTFGCCCGATATTACAAGCCGCAACTTCAACG
TCCGCACGTTCGCCGAGCGGACGGCGATGAACACACCGATCCAGGGATCCGCTGCCGACATCATTAAG
AAAGCGATGATCGATCTAAGCGTGAGCGTGCGCGAAGAACGGCTGCAGGCGCGCCTGTTGCTGCAAG
GTCATGACGAACTCATTTTGGAGGCGCCGAAAGAGGAAATCGGACGGCTGTGCCGCCTCGTTCCGGAA
GTGATGGAGCAAGCCGTGACACTTCGCGTGCCGCTGAAAGTCGATTACCATTACGGTCCGACGTGGTA
CGACGCCAAATAA.
LENGTH: 1992 nucleotides
TYPE: nucleic acid
TOPOLOGY: plasmid
NUMBER OF STRANDS: one
TYPE OF THE MOLECULAR: DNA

1. A fusion polymerase of single-stranded DNA polymerase Bst or another polymerase of this class of DNA polymerases connected with NeqSSB protein or a protein with a sequence similar to NeqSSB in the degree not higher than 50% at N-end of the polymerase using a linker of an exemplary amino acid sequence Gly-Ser-Gly-Gly-Val-Asp or fused directly without a linker, wherein the mentioned polymerase is present in three different variants.

2. Fusion The fusion DNA polymerase NeqSSB-Bst according to claim 1, wherein it contains one of the three variants of Bst polymerase:
Full length—whole amino acid sequence of DNA polymerase|Bst with disabled 5′-3′ activity thanks to the point mutation;
Large Fragment—DNA polymerase|Bst without 5′-3′ domain;
Short Fragment—short version with the deletion of both exonucleolytic domains.

3. Fusion The fusion DNA polymerase NeqSSB-Bst according to claim 1, wherein it binds to all types of DNA and RNA.

4. Fusion The fusion DNA polymerase NeqSSB-Bst according to claim 1, comprising the sequence presented in SEQ1.

5. The fusion DNA polymerase NeqSSB-Bst according to claim 1, comprising the sequence presented in SEQ2.

6. The fusion DNA polymerase NeqSSB-Bst according to claim 1, comprising the sequence presented in SEQ3.

7. A nucleic acid molecule encoding the fusion DNA polymerase NeqSSB-Bst Full Length presented in SEQ 4, the fusion DNA polymerase NeqSSB-Bst Large Fragment presented in SEQ 5, or the fusion DNA polymerase NeqSSB-Bst Short Fragment presented in SEQ 6SEQ 6.

8-9. (canceled)

10. The nucleic acid molecule encoding the fusion DNA polymerase NeqSSB-Bst according to claim 7.

11. The A preparation method of the fusion DNA polymerase NeqSSB-Bst defined in claim 1 wherein:
a first step includes the expression of the gene encoding the enzyme in the optimised conditions in microbiological shaker: growth temperature 28-37° C., incubation time of the medium after induction—3-20 h, inductor concentration—0.1-1 mM IPTG,
obtained cell lysate is subjected to the disintegration using ultrasound and elimination of the DNA genomic contamination using dsDNase,
a second purification step utilises the metal affinity chromatography with His-Trap beads,
next steps cover triple dialysis (10 mM Tris-HCl pH 7.1, 50 mM KCl, 1 mM DTT, 0.1 mM EDTA, 50% Glycerol, 0.1% Triton X-100), gel filtration, and concentration of the preparation,
all processes were conducted in 4° C.,
the purity of the obtained proteins was tested using SDS-PAGE electrophoresis, and the number of units for the obtained preparation was determined using EvaEZ Fluorometric Polymerase Activity Assay Kit.

12. (canceled)
